(19) United States
(12) Patent Application Publication
(10) Pub. No.: US 2007/0147579 A1
(43) Pub. Date: Jun. 28, 2007

(54) METHOD AND SYSTEM FOR RADIOGRAPHIC IMAGING WITH ORGAN-BASED RADIATION PROFILE PRESCRIPTION

(76) Inventors: Bruno K. B. De Man, Clifton Park, NY (US); Robert Franklin Senzig, Germantown, WI (US)

Correspondence Address: GENERAL ELECTRIC COMPANY;GLOBAL RESEARCH, PATENT DOCKET RM. BLDG. K1-4A59, NISKAYUNA, NY, 12309, US

(21) Appl. No.: 11/275,330

(22) Filed: Dec. 23, 2005

(51) Int. Cl. H05G 1/60; A61B 6/00; G01N 23/00; G21K 1/12

(52) U.S. Cl. 378/16

(57) ABSTRACT

A method and system is disclosed that minimizes the effective dose to an object by determining a segmented component map for the object, parametrizing tube current/energy level/x-ray filtration/x-ray pulse width as a function of time, determining a corresponding absorbed dose map and variance map, and determining an energy level/tube current profile or curve that results in a desirable effective dose to the object and a desirable noise variance throughout the image.

### BACKGROUND OF THE INVENTION

The present invention relates generally to diagnostic imaging and, more particularly, to a method and apparatus for maximizing image quality for an image of a multi-component object while minimizing the absorbed dose by the object on a per-component basis.

Generally, four key properties define the performance of a computed tomography (CT) scan: spatial resolution, temporal resolution, image noise, and radiation dose. Spatial resolution defines the degree of small object detail in an image and is generally affected by a number of factors including detector aperture, number of acquisition views, focal spot size, object magnification, slice thickness, slice sensitivity profile, helical pitch, reconstruction algorithm, pixel matrix, patient motion, and field-of-view. Temporal resolution defines the length of the temporal interval over which the scan data is acquired for a given slice. Generally, it is desirable to increase temporal resolution (i.e., reduce the length of the temporal interval) as it enables improved imaging of anatomy in motion, such as the heart. Image noise is the random error on the reconstructed image pixel values due to quantum noise or electronic noise, and largely depends on scan geometry and protocol, patient-anatomy, and is location dependent. Radiation dose corresponds to the number of x-rays absorbed by the patient during a scan.

There is an increasing desire to reduce radiation dose to a patient during radiographic data acquisition. However, since quantum noise level is inversely proportional to the square root of the number of x-rays, image quality is directly related to radiation exposure. That is, image quality generally improves as higher radiation doses are used for data acquisition. Over the years, radiation profiles have become more and more optimized. Radiation dose is modulated spatially by the use of bowtie filters, resulting in decreased radiation towards the periphery of the field of view to compensate for the reduced path lengths thereat. Radiation dose is modulated temporally by using tube current modulation, resulting in decreased radiation at view angles and z-position where the path lengths are smaller, for example, lower radiation anterio-posterior relative to laterally, or, for example, lower radiation in the head region and higher radiation in the shoulder region. Finally, the energy profile is optimized for a given application by choosing an optimal tube voltage and hardware filtration.

Since some organs are more sensitive than other organs, it is desirable to limit irradiation to sensitive organs as much as possible, for example, minimizing the absorbed dose to the thyroid, the breasts, the eyes, etc. Sensitive anatomical structures generally comprise only a portion of a given region-of-interest of which an image is to be reconstructed. Thus, if the radiation dose is set to the maximum permitted for the sensitive anatomical structures, the entire image will have poor spatial and contrast resolution. In this regard, the radiation experienced by a patient varies during the course of the scan. This variable radiation profile is typically achieved via x-ray tube current modulation, x-ray tube voltage modulation, x-ray pulse width modulation, x-ray filter modulation, x-ray tube focal spot modulation, or a combination thereof.

In conventional CT scans, the variable radiation dose profile is constructed so as to minimize the variance (noise in image) for a given amount of radiation, or vice-versa. In other words, in conventional CT scans, the radiation profile used to define the scan considers the total radiation, but does not consider the effective dose for the patient. That is, conventionally, the optimal radiation profiles for given acceptable noise variances and the manner for achieving those optimal radiation profiles for the several anatomical structures that comprise a given region-of-interest are not considered.

Therefore, it would be desirable to design an apparatus and method for tailoring a radiation dose profile to optimize the radiation dose on a per component structure basis while maintaining image noise below a noise variance level.

### BRIEF DESCRIPTION OF THE INVENTION

The present invention is directed to a dose optimization process that overcomes the aforementioned drawbacks. The present invention includes a methodology to find a spatial and temporal radiation profile that results in a desirable trade-off between image quality and effective patient dose. The effective dose to an object is minimized by determining a segmented component map for the object, parameterizing tube current/energy level/x-ray filtration/x-ray pulse width as a function of time, determining a corresponding absorbed dose map and variance map, and determining an energy level/tube current profile or curve that results in the lowest effective dose to the object for a given constraint on the noise variance, or vice-versa. Therefore, in accordance with an aspect of the invention, an imaging system is disclosed as having a computer that executes a computer program representing a set of instructions that when executed by the computer causes the computer to determine a component map of an object to be imaged. The object has a plurality of identifiable and imageable components. The computer also determines a relationship between coefficients of a radiation profile and resulting effective dose for the object and also determines a relationship between the coefficients of the radiation profile and a measure of the resulting variance in an image of the object. The computer further determines an irradiating profile that results in one of a minimal effective dose for the object without noise in an image of the object exceeding a desired noise variance, a minimal noise variance for an image of the object for a desired effective dose, or a desired effective dose for the object and a desired noise variance for an image of the object without total dose to the object exceeding a prescribed limit and noise in an image of the object not exceeding a noise limit.

In accordance with another aspect, a radiographic imaging system is presented and includes an x-ray source configured to project x-rays towards a detector according to a certain radiation profile, which establishes number of x-rays projected and energy level of the x-rays projected as a function of time and location, and possibly a finite time interval during which x-rays are produced for each view. The detector is configured to output electrical signals in response to a reception of x-rays. The system further has a computer programmed to acquire an organ map for a subject to be imaged and determine a parameterized dose absorption map for the subject to be imaged and determine a parameterized noise variance map for the subject to be imaged. The computer further determines an irradiation profile that minimizes effective dose for each organ of the organ map and maximizes image quality for an image of the subject.

According to another aspect, a method of dose management for a CT scan is disclosed. The method further includes the step profiling anatomical layout of a patient to be scanned wherein the object has a plurality of anatomical structures. The method also includes the steps of determining a relationship between coefficients of a radiation profile and an absorbed dose for each of the plurality of anatomical structures and determining a relationship between the coefficients of the radiation profile and a noise variance for an image of the patient. The method then determines a radiation profile that results in each anatomical structure receiving a minimal radiation dose without exceeding a noise variance for the image of the patient.

Various other features and advantages of the present invention will be made apparent from the following detailed description and the drawings.

### BRIEF DESCRIPTION OF THE DRAWINGS

The drawings illustrate one preferred embodiment presently contemplated for carrying out the invention.

In the drawings:

FIG. 1 is a pictorial view of a CT imaging system.

FIG. 2 is a block schematic diagram of the system illustrated in FIG. 1.

FIG. 3 is a schematic illustrating a dose optimization strategy according to the present invention.

FIG. 4 illustrates an exemplary attenuation map.

FIG. 5 illustrates an exemplary absorbed dose map.

FIG. 6 illustrates an exemplary segmented component map.

FIG. 7 illustrates an exemplary noise variance map.

FIG. 8 illustrates application of a well-tailored radiation profile for sensitive organ imaging.

### DETAILED DESCRIPTION OF THE PREFERRED EMBODIMENT

The operating environment of the present invention is described with respect to a four-slice computed tomography (CT) system for imaging of a multi-component object, such as a medical patient. However, it will be appreciated by those skilled in the art that the present invention is equally applicable for use with single-slice or other multi-slice configurations. Moreover, the present invention will be described with respect to the detection and conversion of x-rays. However, one skilled in the art will further appreciate that the present invention is equally applicable for the detection and conversion of other types of radiation. The present invention will be described with respect to a “third generation” CT scanner, but is equally applicable with other CT systems. For example, the invention is also applicable with systems having multiple source spots for increased flexibility in determining an optimal radiation profile by individually steering the different sources.

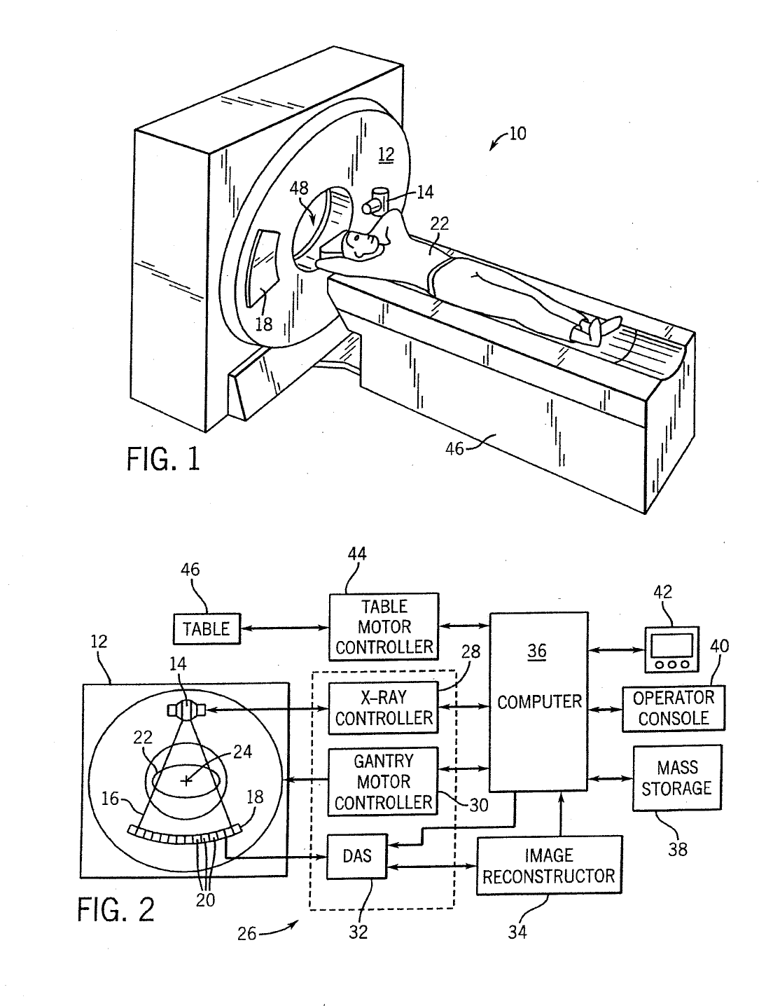

Referring to FIGS. 1 and 2, a computed tomography (CT) imaging system 10 is shown as including a gantry 12 representative of a “third generation” CT scanner. Gantry 12 has an x-ray source 14 that projects a beam of x-rays 16 toward a detector array 18 on the opposite side of the gantry 12. Detector array 18 is formed by a plurality of detectors 20 which together sense the projected x-rays that pass through a medical patient 22. Each detector 20 produces an electrical signal that represents the intensity of an impinging x-ray beam and hence the attenuated beam as it passes through the patient 22. During a scan to acquire x-ray projection data, gantry 12 and the components mounted thereon rotate about a center of rotation 24.

Rotation of gantry 12 and the operation of x-ray source 14 are governed by a control mechanism 26 of CT system 10. Control mechanism 26 includes an x-ray controller 28 that provides power and timing signals to an x-ray source 14 and a gantry motor controller 30 that controls the rotational speed and position of gantry 12. A data acquisition system (DAS) 32 in control mechanism 26 samples analog data from detectors 20 and converts the data to digital signals for subsequent processing. An image reconstructor 34 receives sampled and digitized x-ray data from DAS 32 and performs high speed reconstruction. The reconstructed image is applied as an input to a computer 36 which stores the image in a mass storage device 38.

Computer 36 also receives commands and scanning parameters from an operator via console 40 that has a keyboard. An associated cathode ray tube display 42 allows the operator to observe the reconstructed image and other data from computer 36. The operator supplied commands and parameters are used by computer 36 to provide control signals and information to DAS 32, x-ray controller 28 and gantry motor controller 30. In addition, computer 36 operates a table motor controller 44 which controls a motorized table 46 to position patient 22 and gantry 12. Particularly, table 46 moves portions of patient 22 through a gantry opening 48.

The present invention is directed to a process for determining a dose profile that minimizes the effective dose for a certain image quality or optimizes image quality for a given effective dose. For purposes of this application reference will made to mA/kV modulation which establishes the manner in which the x-ray tube is controlled to produce a desired number of x-rays and an energy level for those x-rays as a function of view angle and position. However, it is contemplated that other factors in addition to the energization of the x-ray tube may help define the radiation dose to an object, such as degree and type of x-ray filtration and the length of time a focal spot of a multi-focal spot x-ray tube is energized. Therefore, reference to mA/kV includes the radiation profile that defines the irradiation experienced by a subject as a result of tube current, tube voltage, x-ray filter filtration, focal spot energization, and the like.

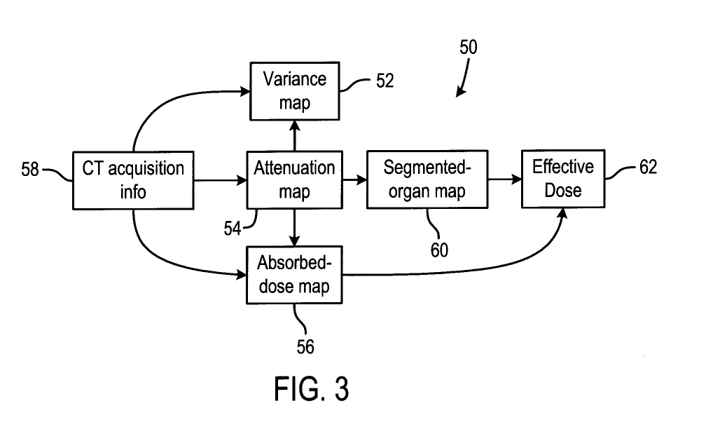

Referring now to FIG. 3, an overview of the mA/kV modulation optimization process according to the present invention is shown. The process 50 determines an effective dose by combining information gathered from a noise variance map 52, an attenuation map 54, and an absorbed dose map 56. As will be described in greater detail below, the noise variance map 52 and the absorbed dose map 56 are derived from CT acquisition information 58 and the attenuation map 54. The CT acquisition information 58 refers to a radiation mA/kV profile that is to be optimized. The attenuation map 54 is also used to derive a segmented component map 60 which together with the absorbed dose map 56 is used to derive an effective dose formula 62. In this regard, the effective dose formula 62 can be used to determine the effective dose for a given set of acquisition parameters 58, and the noise variance formula 52 can be used to determine a noise measure characteristic of the image for a given set of acquisition parameters 58. Similarly, the combination of the effective dose formula 62 and the variance formula 52 can be used to determine the set of acquisition parameters that minimize the effective dose for a given variance in the image, or to minimize the variance in the image for a given effective dose. Further, it is contemplated that rather than minimizing dose and variance relative to one another, the radiation profile can be determined that results in dose and noise being independently constrained such that the relative importance of dose and noise are considered rather than one being minimized at the expense of the other.

Spatial resolution, temporal resolution, image noise, and radiation dose are key parameters for a CT scan. These key parameters can be related to another in the following expression:
σ_{img}˜1/sqrt(D·FWHM³·ST) (Eqn. 1),

where σ_{img} is the standard deviation of the image noise, and D is the radiation dose, FWHM is the full-width-at-half-maximum of the in-plane image point-spread-function, and ST is the slice thickness. While this is a fundamental relationship, the proportionality constant depends strongly on scanner design and efficiency, on the scan protocol, and on the reconstruction technique. Thus, process 50 described above is designed to optimize the number and energy of x-rays generated as a function of time, location, and energy. Thus, for a given scan geometry, an mA value may be established for each view acquisition. For example, for 1000 views, 360 degree acquisition, a radiation value may be established for views 1, 2, 3 . . . 1000. It is recognized that there are some constraints on establishing the radiation values for each view. For example, the radiation settings for each view will be constrained by a maximum value, mA_{MAX}. A parameterized radiation model is then used to compute a dose and a variance map as a function of any possible radiation profile in order to optimize the radiation profile. Therefore, the radiation profile can be modeled as a function of time by the following expression:
mA(τ)=c₁·F₁(τ)+c₂·F₂(τ)+ . . . +c_{N}·F_{N}(τ) (Eqn. 2),

where Fᵢ is a basis function for the mA as a function of time time τ and cᵢ is the weight corresponding to this basis function. One skilled in the art will appreciate that by limiting the radiation profile to a fixed number of basis functions Fᵢ, the computational requirements to determine an optimal radiation profile is less demanding because the number of coefficients cᵢ is typically much smaller than the number of views. For example, by using a basis function that constrains tube modulation to operate along a sine curve and a cosine curve, the number of coefficients is limited to two. One skilled in the art that a multitude of coefficients may be used, but the number may be constrained by the physical limitations of the x-ray tube and/or x-ray filter. That is, a fixed number of different tube current modulations may be permitted by the physics of the x-ray tube and/or x-ray filter and, as such, limit the number of coefficients that are considered for the radiation profile. Equation 2 provides a generalized radiation modulation scheme for an exemplary CT system, such as that shown in FIGS. 1-2. One skilled in the art will also see that Eqn. 2 can easily be generalized to model the cases with multiple sources and to model not only temporal but also spatial or energy modulation.

Referring again to FIG. 3, the effective dose formula 62 and the variance formula 52 are used to optimize dose and image noise for a scan. In this regard, the operator may establish a desired effective dose and a maximum noise variance for the entire scan whereupon the CT system iteratively or empirically derives values for the weight coefficients in Eqn. 2 that will result in an effective dose that does not exceed desired dose while simultaneously providing an image quality within a desired noise variance. Or, conversely, the operator may select a desired maximum noise variance and a desired effective dose whereupon the CT system determines a radiation profile that satisfies, if possible, both the maximum noise variance and the effective dose constraints. If the computational values are found to not be possible to meet the constraints desired by the user, the system preferably conveys that information to the operator to allow the operator to ease the image quality and/or effective dose constraints. In either case, both desirables are considered while establishing a radiation profile for the scan thereby optimizing image quality and effective dose. The radiation profile is not only used to control x-ray tube current and voltage as a function of view angle but is also used to control the degree and manner of x-ray filtration by an x-ray filter if the CT system is equipped with a modulatable x-ray filter.

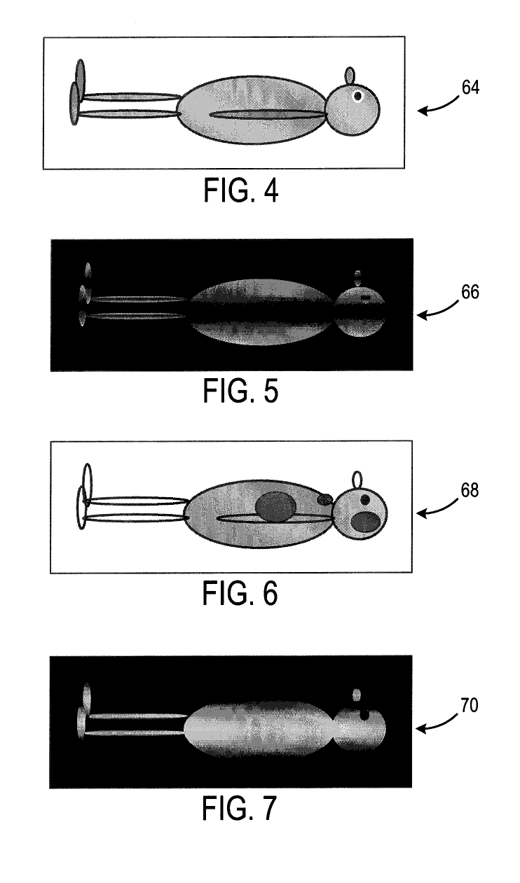

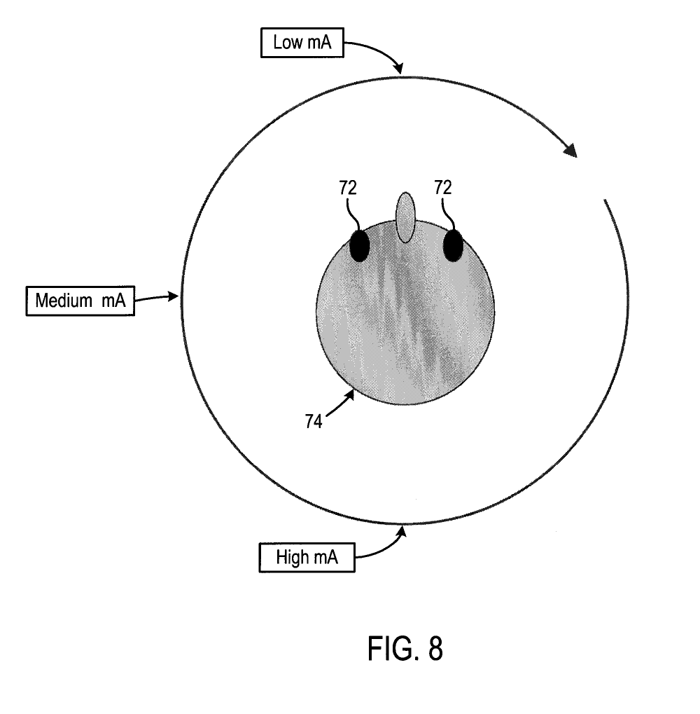

Referring now to FIG. 4, the optimization process of the present invention determines an attenuation map for the object. As illustrated, the attenuation map 64 illustrates the x-ray attenuation pattern for the object. This attenuation map takes into account object density, linear attenuation coefficients, photo-electric attenuation, Compton scatter, etc., for the object to be scanned. The attenuation map may be a 2D or a 3D map and, as described above, is used to derive the absorbed dose map, the noise variance map, and the segmented component map. The attenuation map may be derived from a CT scan, such as a low dose pre-scan, an atlas of general object composition, external markers (position of object ends), object information (height, weight, age, etc.), a radiographic scout scan, a localizer scan, a non-CT scan, or a combination thereof.

Shown in FIG. 5 is an absorbed dose map 66 for the object of FIG. 4. The absorbed dose map is derived from the radiation profile 58 and the attenuation map 64. It is contemplated that a number of known dose absorption tools may be used to derive the absorbed dose map from the radiation profile and the attenuation map. For example, an x-ray tracing method or a detailed Monte Carlo simulation including multiple scatter, energy dependence, etc., is contemplated. As illustrated in the figure, most of the dose is absorbed near the surface of the object nearest the source of x-rays.

Referring now to FIG. 6, a segmented component map 68 is illustrated. Map 68 is derived from the attenuation map 64 using manual or automated segmentation. Map 68 provides a segmentation of the various components of the object to be imaged. In the context of patient imaging, the segmented component map provides a mapping of the patient's organs. Thus, the thyroid, the lungs, the eyes, etc., can be distinguished from one another. This allows for the identification of the location of sensitive and non-sensitive organs of the patient. Instead of or in addition to the attenuation map, an atlas of general object composition, external markers, a scout or other pre-scan, such as a localizer scan, and component particulars, such as height and weight, may also be used to locate the various components of the object. In a preferred embodiment, a standard atlas is warped to provide a clear representation of the specific object's composition.

As set forth with respect to FIG. 3, the attenuation map is used to derive the segmented component map. The component map together with the absorbed dose map is used to determine an effective dose. The effective dose is conventionally defined by the following expression:
Effective Dose= [symbol] ᵢ wᵢ·Dᵢ (Eqn. 3),

where Dᵢ is the average absorbed dose in component i and wᵢ is the weight that is associated with component i. More dose sensitive components are given a higher weight and x-rays to these components will therefore contribute to a larger increase in effective dose. The sum of the weights is assumed to be one. The effective dose is a single value that is desirably minimized and is determined based on the absorbed dose map and the segmented component map.

As shown in FIG. 3, the optimization process also utilizes a noise variance map. An exemplary noise variance map 70 is illustrated in FIG. 7. The noise variance map 70 provides a record of the impact the quantum nature of x-rays have on acquired data. This quantum nature propagates into a variance in the reconstructed image and therefore impacts image quality. The image noise can be determined analytically or numerically based on the noise in the acquired data. Thus, the noise can be determined from projection data (sinogram) of a simulated scan. Accordingly, the attenuation map and the radiation profile are again used as noise is location-dependent. The variance on the image value □ can be defined as E<(□−E<□>)²> where E<> is the expected value. The standard deviation □ is the square root of the variance.

The effective dose formula together with the noise variance map can then be used to optimize dose and image quality on a per-component, per location basis. That is, image noise σ and effective dose D can be calculated as a function of cᵢ or mA(t). Thus, the optimization process can determine D(cᵢ) and σ(x, cᵢ) for a location x. As a result, a constraint can be defined such that σ(x, cᵢ) must be lower than a predefined limit, σₗᵢₘ, in a certain region x ε R and find the cᵢ that minimizes D(cᵢ). On the other hand, the optimization process can similarly require D(cᵢ) to be lower than Dₗᵢₘ and thus minimize the average σ(x, cᵢ) in a certain region x ε R. For example, the result of the optimization process can be a parmetric formula such as D=Σᵢαᵢ while the noise calculation at the center of the image results in α=Σᵢ βᵢ·exp(cᵢ·γᵢ), where αᵢ, βᵢ, and γᵢ are calculated constants that depend on object composition and scanner geometry, and cᵢ are the coefficients to be chosen in an optimal fashion to minimize D and/or σ.

As a result of the described optimization process, an effective dose profile can be determined for a given noise variance, or vice versa. In the context of medical imaging, the invention advantageously determines a mA/kV/filtration profile that takes into account the anatomical weightings that differentiate sensitive and non-sensitive organs. Thus, sensitive organs can be imaged with the minimum dose required to provide an image with the desired noise variance. As a result, as shown in the schematic of FIG. 9, the eyes 72 of a given patient 74 can be imaged in such a manner to limit radiation exposure without introducing unexpected noise into the image. For example, the x-ray tube and x-ray filter may be controlled during their rotation around the patient such that when the x-ray source is above the eyes reduced levels of radiation impinge upon the eyes compared to when the x-ray source is positioned at the side or below the patient. In this regard, radiation exposure will controlled to be greater when the x-ray source is adjacent to non-sensitive regions of the patient compared to when the x-ray source is adjacent to more sensitive regions.

It is contemplated that the present invention can be used singly or in combination with other dose reduction tools to not only limit radiation exposure to a scan subject but also advantageously prevent detector saturation for those types of detectors that easily saturate in a CT scan, such as photon counting and energy discriminating detectors. Thus, the invention may be used with active filter control techniques that dynamically adjust the degree and shape of filtration during the course of a scan to tailor radiation to the given scan subject so as to reduce dose to the subject as well as prevent detector saturation by non-attenuated or reduced attenuated x-rays.

While the present invention has been described with respect to a “third generation” CT scanner, it is contemplated that the invention is also applicable with other radiographic systems. For example, the invention is equivalently applicable with CT scanners having a rotatable x-ray source and a stationary ring of detectors. Moreover, the invention is applicable with so-called “cine CT” scanners having a stationary ring of detectors and a tungsten ring to generate an imaging electron beam. Further, the invention is applicable with helical CT scanners as well as scanners having multiple detector arrays and/or multiple x-ray sources.

Therefore, in accordance with an embodiment of the invention, an imaging system is disclosed as having a computer that executes a computer program representing a set of instructions that when executed by the computer causes the computer to determine a component map of an object to be imaged. The object has a plurality of identifiable and imageable components. The computer also determines a relationship between coefficients of a radiation profile and resulting effective dose for the object and also determines a relationship between the coefficients of the radiation profile and a measure of the resulting variance in an image of the object. The computer further determines an irradiating profile that results in one of a minimal effective dose for the object without noise in an image of the object exceeding a desired noise variance, a minimal noise variance for an image of the object for a desired effective dose, or a desired effective dose for the object and a desired noise variance for an image of the object without total dose to the object exceeding a prescribed limit and noise in an image of the object not exceeding a noise limit.

In accordance with another embodiment, a radiographic imaging system is presented and includes an x-ray source configured to project x-rays towards a detector according to a certain radiation profile, which establishes number of x-rays projected and energy level of the x-rays projected as a function of time and location, and possibly a finite time interval during which x-rays are produced for each view. The detector is configured to output electrical signals in response to a reception of x-rays. The system further has a computer programmed to acquire an organ map for a subject to be imaged and determine a parameterized dose absorption map for the subject to be imaged and determine a parameterized noise variance map for the subject to be imaged. The computer further determines an irradiation profile that minimizes effective dose for each organ of the organ map and maximizes image quality for an image of the subject.

According to another embodiment, a method of dose management for a CT scan is disclosed. The method further includes the step profiling anatomical layout of a patient to be scanned wherein the object has a plurality of anatomical structures. The method also includes the steps of determining a relationship between coefficients of a radiation profile and an absorbed dose for each of the plurality of anatomical structures and determining a relationship between the coefficients of the radiation profile and a noise variance for an image of the patient. The method then determines a radiation profile that results in each anatomical structure receiving a minimal radiation dose without exceeding a noise variance for the image of the patient. The present invention has been described in terms of the preferred embodiment, and it is recognized that equivalents, alternatives, and modifications, aside from those expressly stated, are possible and within the scope of the appending claims.

1. An imaging system having a computer that executes a computer program representing a set of instructions that when executed by the computer causes the computer to:
determine a component map of an object to be imaged, the object having a plurality of identifiable and imageable components;
determine a relationship between coefficients of a radiation profile and resulting effective dose for the object;
determine a relationship between the coefficients of the radiation profile and a measure of the resulting noise variance in an image of the object; and
determine an irradiating profile that results in images obtained having one of a minimal effective dose for the object without noise in an image of the object exceeding a desired noise variance, a minimal noise variance for an image of the object for a desired effective dose, or a desired effective dose for the object and a desired noise variance for an image of the object without total dose to the object exceeding a prescribed limit and noise in an image of the object not exceeding a noise limit.

2. The system of claim 1 wherein the computer is further programmed to assign a radiation dose weight to each component such that a sum of all weights equals one.

3. The system of claim 1 wherein the computer is further programmed to execute one of a scout scan and a localizer scan and determine the component map therefrom.

4. The system of claim 3 wherein the one of a scout scan and a localizer scan is a low dose scan.

5. The system of claim 1 wherein the computer is further programmed to determine the component from an atlas generic for a class of subjects of which the object is a member.

6. The system of claim 1 wherein the computer is further programmed to parameterize the irradiating profile as a function of time and location.

7. The system of claim 1 configured as a CT imaging system.

8. The system of claim 87 wherein the CT imaging system has a rotatable gantry that supports an x-ray source and an array of detectors that are rotated around the object during data acquisition.

9. The system of claim 1 wherein the plurality of identifiable and imageable components corresponds to anatomical structures of a patient.

10. The system of claim 9 wherein the computer is further programmed to define the irradiating profile such that sensitive anatomical structures are exposed to less radiation than non-sensitive anatomical structures with noise in an image of the patient not exceeding the desired noise variance or total dose not exceeding the desired effective dose.

11. A radiographic imaging system comprising:
an x-ray source configured to project x-rays towards a detector according to a certain radiation profile, which establishes number of x-rays projected and energy level of the x-rays projected as a function of time and location, the detector configured to output electrical signals in response to a reception of x-rays; and
a computer programmed to:
acquire an organ map for a subject to be imaged;
determine a parameterized dose absorption map for the subject to the imaged and determine a parameterized noise variance map for the subject to be imaged; and
determine an irradiation profile that minimizes effective dose for each organ of the organ map and maximizes image quality for an image of the subject.

12. The system of claim 11 wherein the computer is further programmed to define the irradiation profile as a function of at least one of tube current, tube voltage, x-ray filtration, and focal spot energization time.

13. The system of claim 11 wherein the computer is further programmed to determine an attenuation map and determine the irradiation profile from at least the attenuation map.

14. The system of claim 13 wherein the computer is further programmed to determine the organ map from at least one of a scout scan, a localizer scan, a subject atlas, subject-specific information, and the attenuation map.

15. The system of claim 13 wherein the computer is further programmed to determine the parameterized dose absorption map from at least one of CT acquisition parameters and the attenuation map.

16. The system of claim 13 wherein the computer is further programmed to determine the parameterized noise variance map from at least one of a sinogram, CT acquisition parameters, the attenuation map, and a simulated sinogram.

17. The system of claim 13 wherein the computer is further programmed to determine the attenuation map from at least one of a scout scan, a localizer scan, an atlas, subject-specific information, and a previous CT scan.

18. The system of claim 11 wherein the x-ray source and the detector are rotated around the subject during data acquisition.

19. A method of dose management for a CT scan, the method comprising the steps of:
profiling anatomical layout of a patient to be scanned, the object having a plurality of anatomical structures;
determining a relationship between coefficients of a radiation profile and an absorbed dose for each of the plurality of anatomical structures;
determining a relationship between the coefficients of the radiation profile and a noise variance for an image of the patient; and
determining a radiation profile that results in, each anatomical structure receiving a minimal radiation dose without exceeding a noise variance for the image of the patient.

20. The method of claim 19 further comprising the step of defining the radiation profile as a function of at least one of x-ray tube current, x-ray tube voltage, x-ray filter filtration power, and x-ray tube focal spot exposure time.

21. The method of claim 19 further comprising the step of defining the radiation profile such that radiation sensitive anatomical structures receive less radiation than non-sensitive anatomical structures.
